(19) United States
(12) Patent Application Publication
(10) Pub. No.: US 2003/0161756 A1
(43) Pub. Date: Aug. 28, 2003

(54) Microdispersion treatment of a protein or pharmaceutical

(76) Inventors: Charles M. Heldebrant, Arcadia, CA (US); Stephan B. Abramson, Altadena, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/080,836

(22) Filed: Feb. 22, 2002

(51) Int. Cl. A61L 2/08

(52) U.S. Cl. 422/22; 422/24; 261/115; 250/435

(57) ABSTRACT

A device and method for non-destructively inactivating microorganisms, and more particularly for non-destructively inactivating microorganisms by subjecting a microdispersion of biological or other pharmaceutical material to short-duration, high-intensity pulses of an energetic emission is provided. The microdispersion treatment device comprises an emitter; a power supply coupled to the emitter; a flow chamber designed to produce a microdispersion of the material across an exposure region such that the material is substantially non-destructively exposed to an emission emitted from the emitter.

### FIELD OF THE INVENTION

The present invention relates generally to the inactivation of microorganisms utilizing radiation pulses, and more particularly to the treatment of a microdispersion of biologic or other pharmaceutical materials using high-intensity pulses of broad-spectrum light.

### BACKGROUND OF THE INVENTION

Significant research has been directed at developing systems for treating labile biologic and small molecule organic pharmaceuticals for the purpose of inactivating contaminating microorganisms. A particular need exists for methods and apparatuses for sterilizing or reducing the microbiological contamination in proteins, small molecule organic pharmaceuticals and other materials vulnerable to microbial contamination without damaging the material.

One approach to the problem of microbiological inactivation has been directed to aseptic packaging technology for packaging of sterilized materials in sterile packaging materials, in order to achieve materials free of microbial contamination and having an extended shelf life. However, such methods and apparatuses have various disadvantages, such as requiring the extensive use of chemical disinfectants, which may leave residual chemical contaminants in the material and in some cases may lead to destruction of the biochemically labile material itself.

Another system for treating such biochemically labile materials utilizes microbiocides and microbiostats to prevent the growth of microbiological contaminants. However, recently such microbiocides and microbiostats have come under increased scrutiny by various governmental agencies, and in some cases have been shown to be potential human carcinogens. As a result, several microbiocides and microbiostats that were once commonly applied to the surface of products in order to prolong the shelf-life of the products have been or could be banned by governmental agencies.

Recently the photobiological effects of a broad-spectrum of light based techniques, have been studied. See, e.g., U.S. Pat. Nos. 4,871,559; 4,910,942; and 5,034,235, issued to Dunn et al. (hereinafter, the '559, '942, and '235 patents, respectively), all of which are incorporated herein by reference. However, these efforts have been principally employed to inactivate microorganisms on food products or in containers for food products.

For example, an aseptic packaging system in combination with a photobiological food treatment apparatus has been developed and reported in the '559 patent, which utilizes short pulses of incoherent, broad spectrum light to preserve food products against microbial degradative processes. This system is stated to provide significant shelf-life and stability enhancements to the food product. Application of pulses of high-intensity, incoherent polychromatic light is also stated to provide efficient, effective, high throughput processing and result in many practical and economic advantages. Moreover, the short duration and the spectral range of each pulse is asserted to permit spatial localization of various of the preservative effects of the light pulses to a thin surface layer such as the surface of the food product or packaging material.

Other studies of the photobiological effects of light are reported in Jagger, J., “Introduction to Research in Ultraviolet Photobiology”, Prentice Hall, Inc., 1967. U.S. Pat. No. 2,072,417 describes illuminating substances, e.g., milk, with active rays, such as ultraviolet rays; U.S. Pat. No. 3,817,703 describes sterilization of light-transmissive material using pulsed laser light; and U.S. Pat. No. 3,941,670 describes a method of sterilizing materials, including foodstuffs, by exposing the materials to laser illumination to inactivate microorganisms.

While these systems have proven very effective at treating food products, these systems are generally not applicable to highly labile, potentially microbially contaminated materials, such as proteins and other pharmaceuticals, which can be easily denatured or destroyed by prolonged exposure to the irradiation source. For example, two apparatuses for purifying biological materials utilizing a broad-band light source are described in the '599 patent: a static platform technique in which the sample is placed on a platform or in a container subject to irradiation; and a flow system in which the sample is flowed through a transparent tube, which is itself subject to an irradiation source. However, both of these systems have the disadvantage that the portion of the sample on the wall of the platform or flow-tube is subject to a much greater irradiation intensity than the portion of the internal bulk of the sample. In order to provide sufficient irradiation to effectively decontaminate the entire sample, these boundary regions may be subject to sufficient irradiation to denature or destroy very sensitive biological materials such as proteins or other substances, such as small-molecule organic pharmaceuticals.

Accordingly, there is a need for a system and method of safely and nondestructively treating irradiation-sensitive biochemically labile materials utilizing short-pulses of energetic emissions without the problems of flow-through cells, cell boundary layer effects, cell material energy absorption effects and sample geometry absorption effects that arise from conventional flow-through treatment cells.

### SUMMARY OF THE INVENTION

The present invention is directed generally to a device and method for inactivating microorganisms without significant damage to the sensitive biologic or other organic materials, and more particularly for inactivating microorganisms by subjecting a microdispersion of biological or other pharmaceutical material to short-duration, high-intensity pulses of an energetic emission.

In one embodiment, the invention is directed to an apparatus for inactivating microorganisms in a biological or other pharmaceutical material comprising a flashlamp; a power supply coupled to the flashlamp; a flow chamber designed to produce a microdispersion of the material across an exposure region such that the material is exposed to high-intensity, short-duration pulses of broad-spectrum polychromatic light emitted from the flashlamp, in such a manner that the light destroys microorganisms without damaging the biologic or other pharmaceutical material.

In another embodiment, the flow chamber comprises at least one needle designed and arranged to non-destructively create droplets of the biological or other pharmaceutical material having a substantially uniform size at a substantially uniform rate. In such an embodiment, the droplets created are preferably less than 3 mm in diameter.

In yet another embodiment, the flow chamber further comprises at least one gas nozzle designed to produce a flow of gas along the edges of the flow chamber such that droplets are urged away from the wall by the gas flow. In a preferred embodiment, a chemically inert gas is utilized in the gas flow, such as a noble gas.

In still another embodiment, a droplet retarder is provided such that the rate of fall of the droplets through the flow chamber can be controlled. In one preferred embodiment, the retarder takes the form of an upwardly directed flow of gas. In another preferred embodiment, the retarder is a pair of charged plates designed to adjustably retard the rate of fall of the droplets.

In still yet another embodiment, the flash lamp is designed to emit multiple flashes in a time period such that a droplet falling through the exposure region of the flow chamber is irradiated multiple times. In one such embodiment, a single strobe lamp is utilized. In another such embodiment, a plurality of lamps timed to sequentially emit are utilized. In still another such embodiment, the flash lamp emits in both the ultraviolet and the visible spectra.

In still yet another embodiment, the droplets are collected and any flow gas is removed in a collection chamber positioned at the exit of the flow chamber. In one such embodiment, the droplets are collected on a surface having a low energy coating such as teflon.

In still yet another embodiment, the flow chamber and the collection chamber are temperature controlled to maintain a constant temperature.

In still yet another embodiment, the invention is directed to a method of irradiation treating a microbiologically labile material. The method comprising running a labile biologic or other pharmaceutical material through the microdispersion treatment apparatus described above.

### BRIEF DESCRIPTION OF THE DRAWINGS

The above and other aspects, features and advantages of the present invention will be more apparent from the following more particular description thereof, presented in conjunction with the following drawings wherein:

FIG. 1 is a schematic illustration of one embodiment of an irradiation microdispersion treatment device according to this invention;

FIG. 2 is a schematic illustration of one embodiment of a microdispersion flow chamber according to this invention; and

FIG. 3 is a flow diagram of a process for manufacturing a purified labile biologic or other pharmaceutical material utilizing the irradiation microdispersion treatment device according to this invention.

Corresponding reference characters indicate corresponding components throughout the several views of the drawings.

### DETAILED DESCRIPTION OF THE INVENTION

The present invention is directed to a treatment device and method to nondestructively purify a flow of labile biologic or other pharmaceutical material utilizing an energetic emission without subjecting the sample to the boundary effects inherent in conventional flow cells. It should be understood that the “flow” of material utilized by the current invention refers to a flow or dispersion of small discrete droplets or spheres of the labile biologic or other pharmaceutical material, not a continuous forceful emission, both of which may be encompassed by the general term “spray,” but only the former of which is desired herein, and that such flow is referred to herein as a “microdispersion” and the apparatus as a “microdispersion treatment device.”

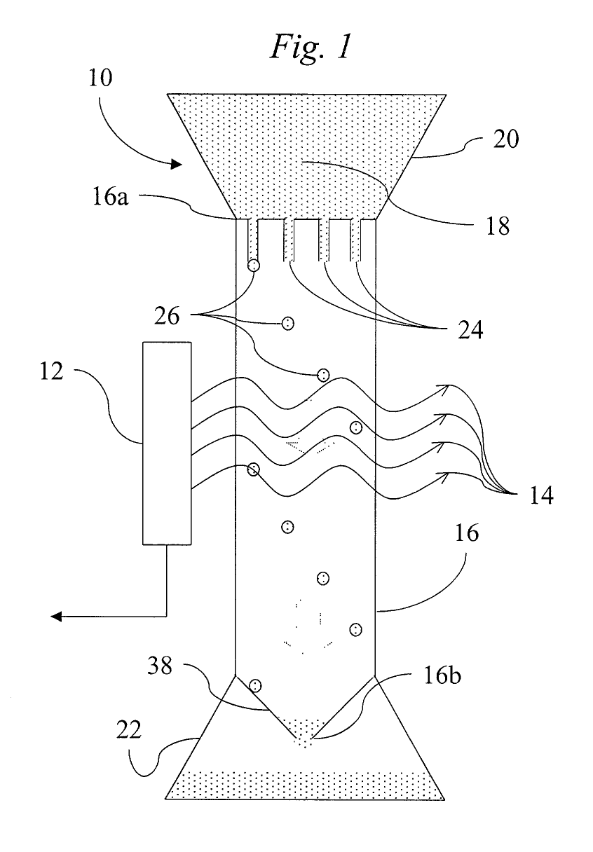

Referring to FIG. 1, a schematic view is shown of an embodiment of an apparatus for the treatment of labile biologic or other pharmaceutical materials such as proteins, genetic materials and pharmaceuticals by exposing a microdispersion of the material to high-intensity, short-duration pulses of an energetic emission. In the embodiment shown in FIG. 1, the microdispersion treatment device 10 comprises an energetic source 12 for emitting an energetic emission 14; an enclosure defining a treatment chamber 16 having an inlet 16a and an outlet 16b through which a microdispersion of the product 18 flows and interacts with the emission 14; a second enclosure defining a pretreatment chamber 20 in fluid communication with the inlet 16a of the treatment chamber 16; and a third enclosure defining a collection chamber 22 in fluid communication with the outlet 16b of the treatment chamber 16.

In the embodiment shown in FIG. 1, the pretreatment chamber 20 comprises a chamber having a plurality of nozzles 24 defining a fluid conduit between the pretreatment chamber 20 and the treatment chamber inlet 16a. In the embodiment shown in FIG. 1, the nozzles 24 are designed such that the product 18 is delivered therefrom into the treatment chamber 16 in the form of a plurality of droplets 26. Any suitable nozzle 24 design may be utilized in the embodiment shown in FIG. 1 such that the droplets 26 are produced without the use of excess shear forces which might damage the material 18, and such that the droplets 26 have a diameter that allows complete penetration of the emission 14 to all droplets 26. For example, the nozzles 24 may comprise a plurality of needles suitably gauged such that the material 18 passes therethrough undamaged. In such an embodiment, it will be understood that the gauge of the needle 24 will depend on the nature of the material to be treated. For example, in a treatment in which the material 18 is an immunoglobulin solution, a large diameter 14 to 20 gauge needle 24 would impart only insubstantial shear forces on the immunoglobulin solution passing therethrough, however, a narrower 30 gauge needle 24 would produce large shear forces and cause substantial damage to the immunoglobulin as the material was forced through the narrow needle shaft. Suitable nozzle diameters are known or easily determined experimentally for any material through conventional strain test assays known in the art.

Although conventional drip nozzles 24 are shown in the embodiment shown in FIG. 1 and described above, any device suitable for non-destructively producing a mist of droplets 26 containing the product 14, and having a diameter suitable for treatment may be utilized in the microdispersion treatment device 10 of the current invention. As discussed above, the droplet formation device 24 chosen has only two requirements: 1) that the chosen method be compatible with the material 18, such that the material is not decomposed, denatured or otherwise damaged by the misting process; and 2) that the diameter of the droplets 26 themselves be suitable for treatment, i.e, such that the emission 14 is capable of sufficiently energizing the entire droplet 26.

Under the first requirement, the method of producing the droplets 26 must be compatible with the material 18 such that the material 18 is not substantially damaged. For example, many labile biological products, such as proteins, are sensitive to shear force and heat. Thus, in an embodiment in which the material 18 to be treated is protein, a droplet device 24 having a low shear force and low heat input should be utilized, e.g., a standard gravity needle drip mechanism as described above or a water-fall mechanism. Likewise, pressurized sources or foaming agents which create large physical and chemical shear forces would not be suitable for such sensitive materials. Generally, materials 18 comprising long chains or materials with a large number of subunits are more easily damaged by shear forces and/or other physical stresses. Techniques suitable for any chosen material 18 are known in the art or are easily determined experimentally via conventional strain test assays.

Under the second requirement, a suitable diameter for the droplets 26 in the device 10 of the invention is one which allows the transmittance of sufficient emission energy through the droplet 26 to inactivate any microbial contaminants in the material 18. A suitable droplet 26 diameter will depend on the concentration of the material 18 and the inherent transmittance of the material 18 to the emission 14 according to Beer's law:

I=I₀e^{-k′cd} (1)

where “d” is equal to the thickness of the material 18 (droplet diameter), “c” equals the concentration of the material 18, “k′” equals the absorption coefficient of the material at a specified emission wavelength and solution temperature, and I₀ equals the initial intensity of the emission 14. According to Beer's law, then, a material having a higher absorption coefficient will require either a lower concentration of solution or a smaller diameter droplet 26 to ensure that the entire droplet 26 is sufficiently energized by the emission 14. Materials such as air and water are relatively transparent to light, including significant portions of the UV spectrum. Accordingly, there is relatively little attenuation through absorption in such media, with the flux density decreasing largely only as a function of distance from the light source. However, for a material having significant absorption, flux density will decrease as a function both of distance from the emitter 12 and of product absorption. Indeed, it will be understood that it may be difficult to treat some highly absorbent materials and/or materials having highly absorbent additives, such as, dyes or detergents or highly scattering additives, such as undissolved particles, because the attenuation of the emission 14 would be too great to provide sufficient exposure to the entire volume for the material. In a preferred embodiment, the droplet diameter is equal to or less than about 3 mm, which corresponds to the utility limit for many treatable substances.

While the intensity of the light penetrating the entire droplet “I” must be sufficient to inactivate micorbial contaminants in the entire volume of the droplet, the level of intensity “sufficient” for the material 18 will depend on the level of inactivating emission energy required for the material and the nature of the material to be irradiated. Such values are either well known from prior studies of the photobiological effects of light or are easily determined experimentally via intensity/inactivation assays. Studies of the degree of efficacy and the required emission intensities have been determined for infrared light (780 to 2600 nm), visible light (380 to 780 nm) and far ultraviolet light (170 to 200 nm). See, e.g., U.S. Pat. Nos. 4,871,559; 4,910,942; and 5,034,235, issued to Dunn et al., all of which are incorporated herein by reference.

In a preferred embodiment of the present invention, the droplet formation device 24 is designed such that droplets 26 of substantially uniform diameter are formed at a substantially uniform rate. Having uniform droplets 26 formed at a uniform rate allows for more reproducible and controlled treatment regiments, i.e., emission intensity, pulse time and dwell time within the treatment chamber to be used reducing the anisotropy of the treatment of the material 18.

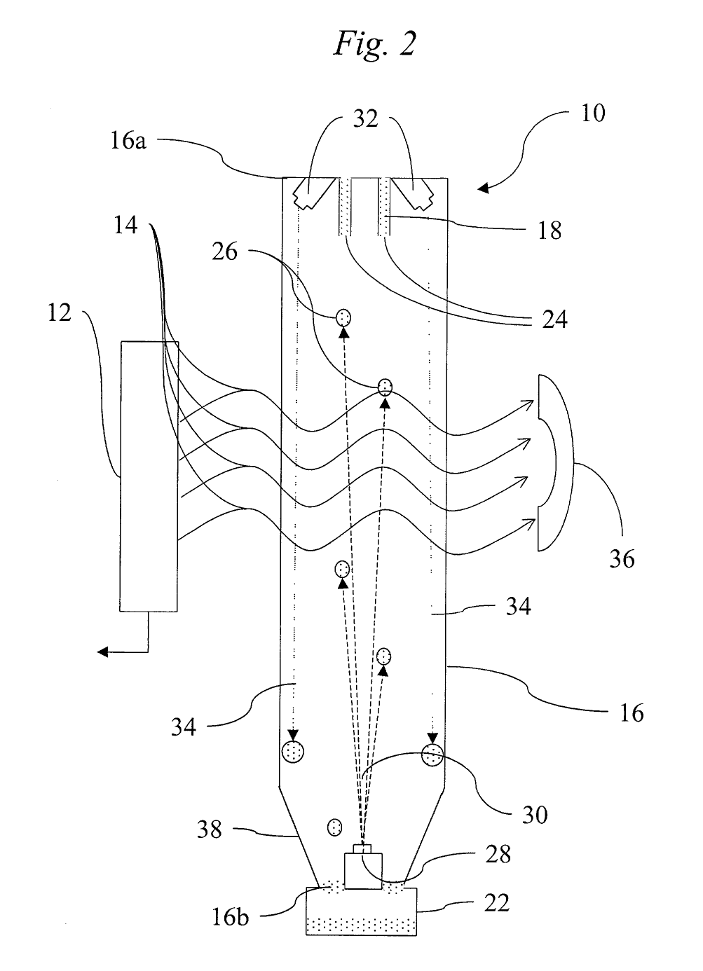

Regardless of the technique utilized to form the droplets 26, the droplets 26 thus formed are dripped through the treatment chamber inlet 16a and into the treatment chamber 16 itself. Although the treatment chamber 16 is shown as a cylinder in the embodiment shown in FIG. 1, any configuration suitable to allow the droplets 26 formed in the pretreatment chamber 20 to move therethrough and interact with the emission 14 impinging on the treatment chamber may be utilized. For example, in one embodiment, shown in FIG. 2, the treatment chamber 16 is rectangular such that the droplets 26 fall through the chamber in a single line, such that the emission 14 is unimpeded prior to impinging on each droplet 26. Regardless of the specific geometry utilized, the dimensions of the treatment chamber 16 will vary depending upon many factors including: the absorption characteristics of the material 18 to be treated, the physical and operating characteristics of the emitter 12, and the rate the material 18 moves through the treatment chamber 16 between pulses, i.e., flashes, of light. To enhance the intensity of the emission 14, the treatment chamber 16 may include a reflector assembly 36 as its outer wall or as an external reflector, in order to reflect the emission 14 traversing the material 18 back into the chamber 16.

The above discussion assumes that the material 18 is allowed to free-fall under the influence of gravity through the treatment chamber 16. However, depending on the total emission exposure required to treat the material 18, and the arrangement of the emission source 12 in relation to the treatment chamber 16, it may be necessary to have an impractically long treatment chamber 16 to ensure that the residence time of the material 18 in the treatment chamber 16 is sufficient or to have an impractically or expensively large number of emitters 12 to ensure that the material 18 is exposed to sufficient energetic emission 14 for treatment. In such cases, a flow controller 28 may be additionally provided in the treatment chamber 16 to control the flow rate of the material 18 through the treatment chamber 16 in relation to the pulse repetition rate of the emission source 12 so that during the residence time of the material 18 within the treatment chamber 16, all of the material 18 is exposed to a predetermined level of emission, i.e., number of pulses, overall intensity of pulses, or overall exposure of material. Accordingly, the flow controller 28 ensures that the material 18 exiting the treatment chamber 16 is treated to a desired degree. In addition, a flow controller 28 ensures uniform transport of the droplets 26 decreasing the anisotropy of the treatment and yielding uniform and reproducible treatment regiments. Alternatively or in addition, the flow controller 28 may provide mixing to insure that all of the material 18 being treated is subjected to an appropriate flux intensity and number of pulses (for the desired degree, or level, of inactivation, i.e., kill or sterilization).

In one exemplary embodiment, shown in FIG. 2, the flow controller 28 comprises at least one gas nozzle designed and arranged to direct a flow of gas 30 upward through the treatment chamber 16 against the falling droplets 26 of material 18 such that the speed of the falling droplets 26 is retarded. In such an embodiment, any gas could be utilized such that the flow of gas 30 does not react with the material 18. For example, suitable chemically inert gases include: argon, helium, neon and nitrogen. If a gas flow 30 is utilized, the gas may be scrubbed of material 18 prior to venting or recycling in the collection chamber by a variety of techniques including filtration, and/or condensation. Further if the gas from the gas flow 30 is to be recycled, it can be purified utilizing any known purification technique, such as, for example, filtration, incineration or irradiation.

Although a gas jet flow controller 28 is described above, any suitable flow controller may be utilized in the present invention. For example, the flow controller 28 may comprise a pair of charged plates designed and arranged to emit a retarding field to control the flow rate of the material 18. In addition, although only retarding flow controllers 28 are discussed above, the flow controller 28 might comprise an accelerating field or gas flow to speed the flow of the droplets of material through the treatment chamber. Finally, although only flow controllers having a linear gravity induced geometry are described above, any suitable geometry of flow may be utilized such as, for example, a helical geometry, or a flow induced via a field or force directed in opposition to the force vector of gravity. The only requirement for the flow controller 28 of the present invention is that the force exerted by the flow controller 28 must be of a level sufficient to adequately retard or accelerate the droplets 26 without denaturing or otherwise damaging the material 18 being treated.

To prevent droplets of the material 18 from adhering to the walls of the treatment chamber 16 and receiving an excessive dose of the emission 14, the treatment chamber may also comprise a system for sweeping material 18 from the walls of the treatment chamber 16. Such a sweeping device 32 system could comprise a plurality of gas nozzles, as shown in FIG. 2, designed and arranged to direct a curtain of gas 34 along the walls of the treatment chamber 16 such that any droplets 26 impinging on the walls are swept therefrom and into the outlet 16b of the treatment chamber 16. In such an embodiment, any gas could be utilized such that the gas 34 does not react with the material 18. For example, suitable chemically inert gases include: argon, helium, neon and nitrogen. If a gas curtain 34 is utilized, the gas flow may be scrubbed of material 18 prior to venting or recycling in the collection chamber by a variety of techniques including filtration, and/or condensation. Further if the gas from the gas curtain 34 is to be recycled it can be purified utilizing any known purification technique, such as, for example, filtration, incineration or irradiation.

Although a gas curtain 34 is described in the exemplary embodiment above, any sweeping device 32 or other method suitable for minimizing droplets 26 impinging on the walls of the treatment chamber 16 may be utilized in the present invention. For example, a liquid or energetic emission curtain could be used in place of the gas curtain 34 to sweep the droplets 26 off the wall. Alternatively, the walls of the treatment chamber 16 might be coated with a substance that prevents droplets 26 from coalescing and adhering thereto. Likewise, the treatment chamber 16 may be made of any material suitable to allow sufficient transmission of the emission 14 therethrough. For example, glass may be utilized in visible light applications where glass is transparent to wavelengths in the visible spectrum (between about 380 and 780 nm). However, in an embodiment in which the emission 14 utilizes the UV portion of the spectrum (between about 170 and 200 nm), quartz or a similar material should be utilized which is transparent to UV light. Although only solid walled apparatuses are discussed above, it should be understood that any device suitable for containing the substance within the treatment region may be utilized with the current invention, such as, for example, a containment gas curtain or powered field.

As discussed above, the emitter 12 shown in FIGS. 1 and 2 schematically, may comprise any suitable source of energizing emission capable of inactivating microbial contaminants in the material 18. In one embodiment, the emitter 12 is a pulsed light source, such as those described in U.S. Pat. Nos. 4,871,559; 4,910,942; 5,034,235; 5,658,530; and 5,900,211 issued to Dunn et al.; U.S. Pat. Nos. 5,768,853 and 6,013,918 issued to Bushnell et al.; and U.S. Pat. No. 5,925,885 issued to Clark et al., all of which are incorporated herein by reference. Typical operating parameters for a suitable light source are a flashlamp which emits an emission having a spectrum including wavelengths of at least about 170 to about 2,600 nm, having pulses of duration between about 0.001 and about 100 ms, and having an intensity between about 0.01 and about 50 J/cm².

In such an embodiment, any pulsed light source emitter 12 may be utilized, such as, for example, a high powered Xenon flashlamp. If a flashlamp emitter 12 is utilized, the flashlamp may be constructed in nearly any shape; and similarly, if reflectors 36 are used in conjunction with the flashlamp emitter 12, the reflectors may be made of many different materials in many different geometries to direct pulses of light from the flashlamps to the product to be treated. Suitable reflector designs may be found in “The Optical Design of Reflectors”, Second Edition, William B. Elmer, Published by John Wiley and Sons, Inc., New York, which is incorporated herein by reference. In such an embodiment, the emitter source 12 may use one or a plurality of flashlamp emitters 12 and associated reflectors 36 to create high-intensity, short-duration pulses of incoherent polychromatic light in a broad spectrum in the treatment chamber 16 in which the material 18 to be treated is exposed to the emission 14.

While the embodiment shown in FIG. 2 utilizes a plurality of individual straight flashlamp emitters 12 and reflector elements 36, other arrangements may also be utilized. For example, one or more flashlamp emitters 12 may alternatively (or in addition) be located externally or internally in the treatment chamber 16. A preferred design is shown in FIG. 2, in which the material 18 to be treated is conducted through a treatment chamber 16 made of a transparent material (e.g., a quartz tube). The treatment chamber 16 is positioned along one focus of an elliptical reflector 36 and a flashlamp emitter 12 is positioned along another focus of the elliptical reflector 36. If a plurality of emitters 12 are utilized, such emitters may all be illuminated simultaneously, may be illuminated in pairs, or may be illuminated serially. Preferably, however, the emitters 12 are illuminated simultaneously such that emissions 14 from adjacent emitters 12 cooperate at the boundaries between such emitters 12 in order to assure that a minimum total fluence is achieved over the entire area being treated, which enables the use of lower fluence levels per flash, while preserving a high overall fluence level and a high degree of material 18 treatment.

Although the embodiments of the present invention discussed above include many potential applications for the reduction of viable organism, microbe or virus numbers, or enzymatic activity in the treatment of biologically labile materials employing high-intensity, short-duration, broad-spectrum, polychromatic, incoherent pulses of light, any emission 14 suitable for the treatment, i.e., inactivation of unwanted microbial materials in a sample may be utilized, such as, for example, gamma rays or other energetic emissions. Further, where a light-based emission 14 is utilized, it is preferred to utilize a broad-spectrum output, including near and far ultraviolet light components of the spectrum, such that multiple inactivation pathways may be energized in the target material simultaneously and thereby relatively low fluences may be utilized. For example, even at very high organism densities (up to 1×10⁶ to 1×10⁸), only one or two flashes at an energy density of 0.4 or 0.5 J/cm² per flash (or as low as 0.1 J/cm², or even as low as 0.01 J/cm² or less, depending on the microorganisms to be inactivated) have been reported to result in inactivation, i.e., kill or sterilization or inactivation, of spores, vegetative bacteria, viruses, unwanted DNA and the like.

In any of the above embodiments, an emission-sensitive element (not shown) may be further incorporated into the treatment chamber 16 such that the intensity of the light reaching the droplets 26 is constantly monitored such that a indication of the effectiveness of the treatment can be constantly read-out. Further, the emission-sensitive element may further be connected through a closed-loop feedback control system to the emitter 12 such that if the intensity of the emission 14 impinging on the droplets 26 is insufficient or excessive, i.e., below or above a prescribed threshold, the power delivered to the emitter 12 and thus the intensity of the emission 14 may be increased or decreased accordingly.

The collection chamber 22 may comprise any receptacle suitable to catch and collect a significant proportion of the droplets 26. In the embodiment shown in FIG. 1, the collection chamber comprises a funnel 38 disposed at the outlet 16b of the treatment chamber 16 such that the droplets 26 passing through the treatment chamber 16 impact the sloped sides of the funnel 38 and are directed into the enclosure defining the collection chamber 22. Although the collection chamber 22 shown in FIG. 1 is a simple vessel having no outlets, it should be understood that the collection chamber 22 could have any suitable outlet (valved or unvalved) such that the treated material 18 passing therein may be directed to any additional processing machinery. For materials 18 having low shear force thresholds, it may be necessary to coat the funnel 38 and/or the inner walls of the collection chamber 22 with a low energy material capable of reducing the impact force felt by the falling droplet. Any suitable low-energy material could be utilized which is inert to the material 18 and capable of being applied to the surfaces of the treatment device 10, such as, for example, teflon, polycarbonate or polypropylene.

Additionally, a temperature controller (not shown) may be integrated into pretreatment chamber 20, the treatment chamber 16 or the collection chamber 22 of the microdispersion treatment device 10 of the current invention to either heat or cool the material 18 before, during and/or after treatment to ensure that the material 18 is maintained at a temperature suitable for the material 18.

The present invention is also directed to a method for treating a biologically labile material 18 utilizing the microdispersion treatment device 10 of the current invention. During operation, a material is introduced into the pretreatment chamber 20 from whence it is either gravity-drained or pressurized such that the material 18 is directed through the droplet formation device 24 and into the inlet 16a of the treatment chamber 16 in the form of a microdispersion of droplets 26.

The droplets 26 then in turn flow through the treatment chamber 16. Simultaneously, the emitter 12 is energized to emit an emission 14 which passes into the treatment chamber 16 such that the emission 14 interacts with the droplets 26 of the material 18 passing therethrough. The droplets may be acted on by various forces to improve the treatment characteristics of the microdispersion treatment device 10. For example, in an embodiment having flow controller 28 the droplets 26 may be retarded by an electric field or a gas flow 30 such that the flow of the droplets 26 through the treatment chamber 16 is regulated. In addition in an embodiment having a wall sweeping device 32, droplets 26 impinging on the walls of the treatment chamber 16 may be swept off the walls via a curtain 34 of gas directed thereagainst by the sweeping device 32.

Once the droplets 26 pass through the treatment chamber 16 and out of the outlet 16b they are collected via a collection device 38 and directed into the collection chamber 22 either for final packaging or for further treatment.

### EXAMPLE 1

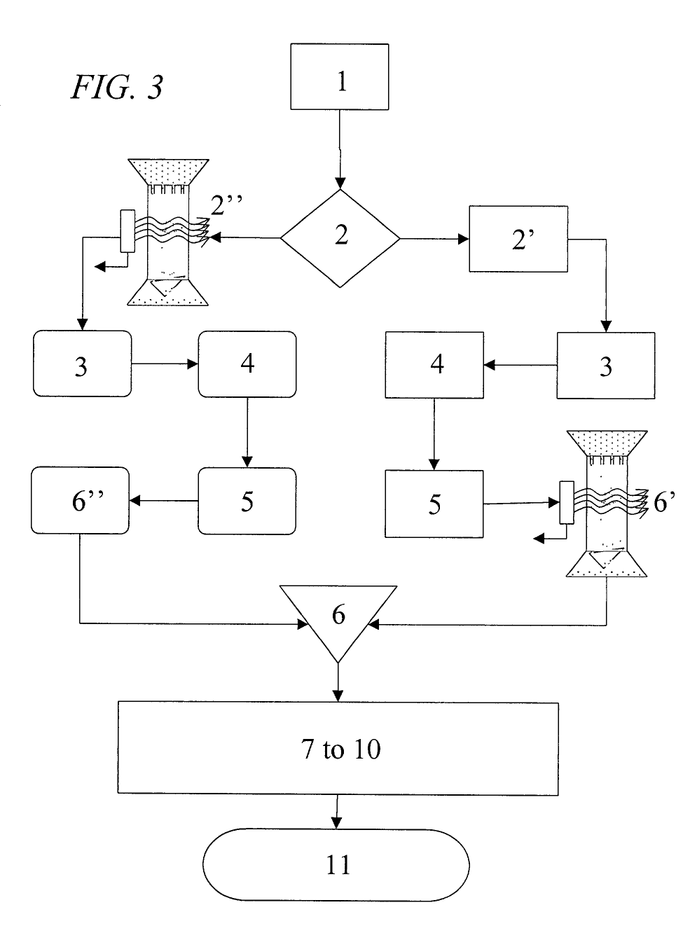

An exemplary embodiment of the treatment of a solution of 5% liquid immune globulin intravenous protein (IGI) utilizing the microdispersion treatment device and method is described below in relation to FIG. 3.

In this hypothetical embodiment, two alternative treatment pathways are shown. For either treatment pathway, Step (1) of the process would involve obtaining a precipitate (GGI-ppt) containing the IGI protein. In Step (2), the precipitate would then be material balanced for clarification. In the process including Step 2′, the material balance would be conducted without any microdispersion treatment, while the process including Step 2″ would run the material balanced IGI through the microdispersion treatment device 10 of the current invention to inactivate any unwanted contaminants. Steps 3, 4 and 5 of either of the processes would involve an OSD treatment followed by the adsorption and filtration of the material, then followed by a CM Sephadex wash, respectively. Step 6 involves a CM Sephadex elution. In the process including Step 6′ the CM Sephadex elution would further involve running the IGI sample through the microdispersion treatment device 10 according to the present invention, while the process including Step 6″, would have already treated the IGI sample by that point. Steps 7 to 10 in such an embodiment would then be identical for the two processes; the IGI would enter a post wash, followed by a pre-UF clarification, followed by an ultra-filtration, followed by the addition of Sorbitol and a pH adjustment, respectively. Finally, at Step 11 the material would be sterilely filtered and prepared for packaging and shipment.

The choice of processing steps at which the microdispersion treatment according to the present invention may be conducted in this hypothetical example depends on the presence of additives in the protein and the relative concentration and volume of the protein. For example, most of the steps of the process diagrammed in FIG. 3 and described above would either contain very high concentrations of the material, or contain additives that would scatter or absorb the emission energy, thereby making them impractical for the microdispersion treatment process of the current invention. However, at Step 6″ of the hypothetical process, no energy absorbing additives would be present in the protein solution and the average protein concentration of the IGI is only 18 mg/ml in an average solution volume of ˜1200L. Although the overall volume would be large, because the concentration of the IGI protein would be low, the level of emission could be decreased and the exposure time could be decreased, while simultaneously allowing for an increase in the droplet size, thereby allowing for very high volume throughput for the microdispersion treatment device.

In contrast, the alternative hypothetical process in which the IGI protein would be treated at Step 2′ would require significantly more vigorous treatment conditions. Here, while no emission absorbing additives would be present in the protein solution and while the total volume of the IGI would be only ˜390L, the concentration of the IGI would be ˜63 mg/ml. Because the concentration of the protein would be high, if the microdispersion treatment is utilized at this stage of the hypothetical process, it would be necessary to run the emitter 12 at higher intensity, and/or provide for longer exposure times of the protein in the treatment chamber 16, and/or create smaller droplet diameters to ensure that sufficient emission exposure is delivered to the entire volume of the protein material.

Although the above discussion describes a process involving the treatment of a protein, it should be understood that biological material or biochemically labile material is meant to encompass any material designed to be administered to a patient by any of several routes, including, but not limited to, intravenous or intramuscular injection, to provide a pharmaceutical effect, such as, for example, proteins, pharmaceuticals, gene therapy products, etc.

While the invention herein disclosed has been described by means of specific embodiments and applications thereof, numerous modifications and variations could be made thereto by those skilled in the art without departing from the scope of the invention set forth in the claims.

What is claimed is:

1. A microdispersion treatment device for treating materials comprising:
at least one emitter coupled to at least one power supply for producing an energetic emission;
an exposure region designed to allow the emission to pass therethrough; and
a droplet formation device designed to deliver a microdispersion of droplets of a liquid solution of material to the exposure region such that the droplets pass through the exposure region and the material is substantially non-destructively exposed to the energetic emission.

2. A microdispersion treatment device as described in claim 1, wherein the droplet formation device comprises at least one needle.

3. A microdispersion treatment device as described in claim 2, wherein the needle has a gauge between about 14 and 30.

4. A microdispersion treatment device as described in claim 1, wherein the droplets are less than about 3 mm in diameter.

5. A microdispersion treatment device as described in claim 1, further comprising at least one gas nozzle arranged within the exposure region and designed to produce a flow of gas along the walls of the exposure region such that droplets are urged away from the walls of the exposure region by the gas flow.

6. A microdispersion treatment device as described in claim 5, wherein the gas is a chemically inert gas.

7. A microdispersion treatment device as described in claim 5, wherein the gas is selected from the group consisting of: a noble gas and nitrogen.

8. A microdispersion treatment device as described in claim 1, further comprising a flow controller arranged within the exposure region and designed such that the residence time of the droplets in the exposure region can be controlled.

9. A microdispersion treatment device as described in claim 8, wherein the flow controller is gas nozzle design to direct a flow of gas at the droplets such that the residence time of the droplets depends on the speed of the flow of gas.

10. A microdispersion treatment device as described in claim 9, wherein the gas nozzle is designed to increase the residence time of the droplets.

11. A microdispersion treatment device as described in claim 9, wherein the gas nozzle is designed to decrease the residence time of the droplets.

12. A microdispersion treatment device as described in claim 9, wherein the gas is a chemically inert gas.

13. A microdispersion treatment device as described in claim 9, wherein the gas is selected from the group consisting of: a noble gas and nitrogen.

14. A microdispersion treatment device as described in claim 8, wherein the flow controller is a pair of charged plates designed to create a charged field within the exposure region such that the residence time of the droplets within the exposure region depends on the properties of the charged field.

15. A microdispersion treatment device as described in claim 14, wherein the charged plates are designed to increase the residence time of the droplets.

16. A microdispersion treatment device as described in claim 14, wherein the charged plates are designed to decrease the residence time of the droplets.

17. A microdispersion treatment device as described in claim 1, wherein the at least one emitter is at least one flash lamp.

18. A microdispersion treatment device as described in claim 17, wherein the at least one flashlamp is designed to emit a pulse of broad-spectrum light.

19. A microdispersion treatment device as described in claim 17, wherein the at least one flashlamp emits an emission having wavelengths in the visible and ultraviolet spectrum.

20. A microdispersion treatment device as described in claim 17, wherein the at least one flashlamp emits an emission having a spectrum including wavelengths of at least about 170 to about 2,600 nm.

21. A microdispersion treatment device as described in claim 17, wherein the at least one flashlamp emits emission pulses of duration between about 0.001 and about 100 ms.

22. A microdispersion treatment device as described in claim 17, wherein the at least one flashlamp emits an emission having an intensity between about 0.01 and about 50 J/cm².

23. A microdispersion treatment device as described in claim 17, comprising at least two flashlamps.

24. A microdispersion treatment device as described in claim 23, wherein the at least two flashlamps emit sequentially.

25. A microdispersion treatment device as described in claim 23, wherein the at least two flashlamps emit simultaneously.

26. A microdispersion treatment device as described in claim 1, wherein the emitter is a gamma ray emitter.

27. A microdispersion treatment device as described in claim 1, further comprising a collection chamber positioned at the outlet of the exposure region to collect the droplets.

28. A microdispersion treatment device as described in claim 27, wherein the surface of the collection chamber has a low surface energy coating.

29. A microdispersion treatment device as described in claim 28, wherein the low surface energy coating is selected from the group consisting of: teflon, polycarbonate and polypropylene.

30. A microdispersion treatment device as described in either claim 5 or 9, further comprising a flow gas filter arranged and designed to remove the gas flow from the exposure region.

31. A microdispersion treatment device as described in claim 1, further comprising a temperature controller arranged to maintain a constant temperature within the exposure region.

32. A microdispersion treatment device as described in claim 1, wherein the material is selected from the group consisting of: proteins, and pharmaceuticals.

33. A microdispersion treatment device as described in claim 1, wherein the droplet formation device is designed to produce a microdispersion of substantially uniform droplets.

34. A microdispersion treatment device as described in claim 1, wherein the droplet formation device is designed to produce a microdispersion of substantially uniform droplets at a substantially uniform rate.

35. A microdispersion treatment device for treating materials comprising:
at least one emitter coupled to at least one power supply for producing an energetic emission; and
a treatment chamber defining an internal volume having an exposure region designed to allow the emission to pass therethrough, the treatment chamber further comprising an inlet in fluid communication with a reservoir of a liquid solution of material arranged on a first side of the exposure region designed to emit a microdispersion of droplets of the material and an outlet arranged on a second side of the exposure region designed to collect the droplets, such that the droplets pass through the exposure region and such that the material is substantially nondestructively exposed to the energetic emission.

36. A microdispersion treatment device for treating materials comprising:
at least one emitter coupled to at least one power supply for producing an energetic emission;
a treatment chamber defining an internal volume having an exposure region designed to allow the emission to pass therethrough, the treatment chamber further comprising an inlet in fluid communication with a reservoir of a liquid solution of material arranged on a first side of the exposure region designed to emit a microdispersion of droplets of the material and an outlet arranged on a second side of the exposure region designed to collect the droplets, such that the droplets pass through the exposure region and such that the material is substantially nondestructively exposed to the energetic emission;
a flow controller arranged within the treatment chamber and designed such that the residence time of the droplets in the treatment chamber can be controlled; and
at least one gas nozzle arranged within the internal volume designed to produce a flow of gas along the walls of the treatment chamber such that droplets are urged away from the walls of the treatment chamber by the gas flow.

37. A method of treating a material comprising:
providing a source of energetic emission;
providing a liquid solution of material;
separating the solution of material into droplets and exposing the droplets to the energetic emission.

38. A method of treating a liquid material comprising running a solution of material through the microdispersion treatment device as described in claim 1.
